(19) United States
(12) Patent Application Publication
(10) Pub. No.: US 2005/0159696 A1
(43) Pub. Date: Jul. 21, 2005

(54) Tapered tubing for use in extracorporeal circuit for peripheral vein fluid removal

(76) Inventors: Steven Bernard, Andover, MN (US); Kelley Stickan, Hugo, MN (US); Mark Gelfand, New York, NY (US)

Correspondence Address: NIXON & VANDERHYE, PC, 901 NORTH GLEBE ROAD, 11TH FLOOR, ARLINGTON, VA, 22203, US

(21) Appl. No.: 10/760,476

(22) Filed: Jan. 21, 2004

(51) Int. Cl. A61M 37/00

(52) U.S. Cl. 604/6.16

(57) ABSTRACT

A tapered extracorporeal blood tube having at least a first end of the tube with a large diameter, a narrow diameter section of the tube, and a tapered tube transition section between the first end and the narrow section. The tapered blood tube reduces the overall blood flow volume in the circuit.

### BACKGROUND OF THE INVENTION

The invention relates to extracorporeal blood circuits and blood passages in those circuits. In particular, the invention relates to tapered blood tubing in an extracorporeal circuit to minimize blood volume and improve connection with various circuit components.

Extracorporeal blood circuits withdraw blood from the vascular system of a patient, treat the blood, and infuse the treated blood into the patent. Blood tends to clot shortly after being removed from the patient. Coagulation of blood causes blood to form clots that obstruct blood circuits and can harm patients if infused into their vascular systems. Typically, an anticoagulation drug is injected into the extracorporeal blood to reduce coagulation in a blood circuit. There is a desire to eliminate or at least reduce the need for anticoagulation drugs injected into extracorporeal blood circuits.

Coagulation of blood in an extracorporeal blood passage may be activated by various factors including shear of the blood flow, and contact between the blood and the surfaces of the extracorporeal circuit. Once coagulation is activated, clots form in the blood within a few minutes. If the extracorporeal circuit is of a sufficiently small volume and the blood velocity in the circuit is sufficiently great, the blood flows through the entire circuit and is infused back into the patient before clots form even if some coagulation is activated in the circuit. Clotting can be voided or at least substantially reduced by minimizing the residence time of blood in a blood circuit.

Minimizing the volume of the blood flow passage in an extracorporeal circuit generally reduces the amount of time the blood is in the extracorporeal circuit for a given blood flow rate. Minimizing the blood passage volume also reduces the volume of blood lost if a clot forms to block the circuit (and cause the blood flow to cease and the circuit filled with blood to be discarded).

Flow passages through a blood circuit have been developed to minimize coagulation in the circuit. Prior examples of such extracorporeal blood circuits are show in, for example, U.S. Pat. Nos. 6,533,747 and 6,272,930 and Published U.S. Patent Application 2002-0085951. The circuits disclosed in these patent publications have blood passages that are of a constant diameter throughout the length of the circuit. Maintaining a constant diameter and shape of the blood passage throughout the circuit promote laminar blood flow through the circuit and minimizes dead zones in the blood passage where blood flow stagnates and forms eddy currents. Accordingly, conventional wisdom teaches using constant diameter blood tubes to reduce dead zones where blood can clot.

### BRIEF DESCRIPTION OF THE INVENTION

An extracorporeal circuit has been developed having tapered blood passages that are substantially free of obstructions and dead zones. A tapered blood tube reduces the blood passage volume of the circuit, by reducing the cross-sectional area of portions of the blood tube. Reducing the volume of blood in an extracorporeal blood circuit minimizes coagulation by reducing the residence time of blood in the circuit. Tapered blood tubing also has an expanded end(s) to connect to other components in the blood circuits that have relatively large diameter tube connection ports. The circuit can be used, for example, in a blood circuit for continuous filtration of blood using peripheral venous blood access to treat congestive heart failure (CHF), with minimal or no anticoagulation.

In one embodiment, the extracorporeal circuit utilizes tapered tubing to minimize the total circuit volume. Tapered tubing can be sized to fit its mating components at either end of the tubing. The tapered tubing may be sized to have a relatively small diameter at a center section of blood circuit passage, and expand towards the inlet and outlet of the component.

### BRIEF DESCRIPTION OF THE DRAWINGS

FIG. 1 is a longitudinal side-view of tapered tubing for a blood circuit.

FIG. 2 is a longitudinal cross-sectional view of the tapered tubing.

FIG. 3 is a longitudinal cross-sectional view of the tapered tubing showing the tubing attached on one end to male leur connector.

### DETAILED DESCRIPTION OF THE INVENTION

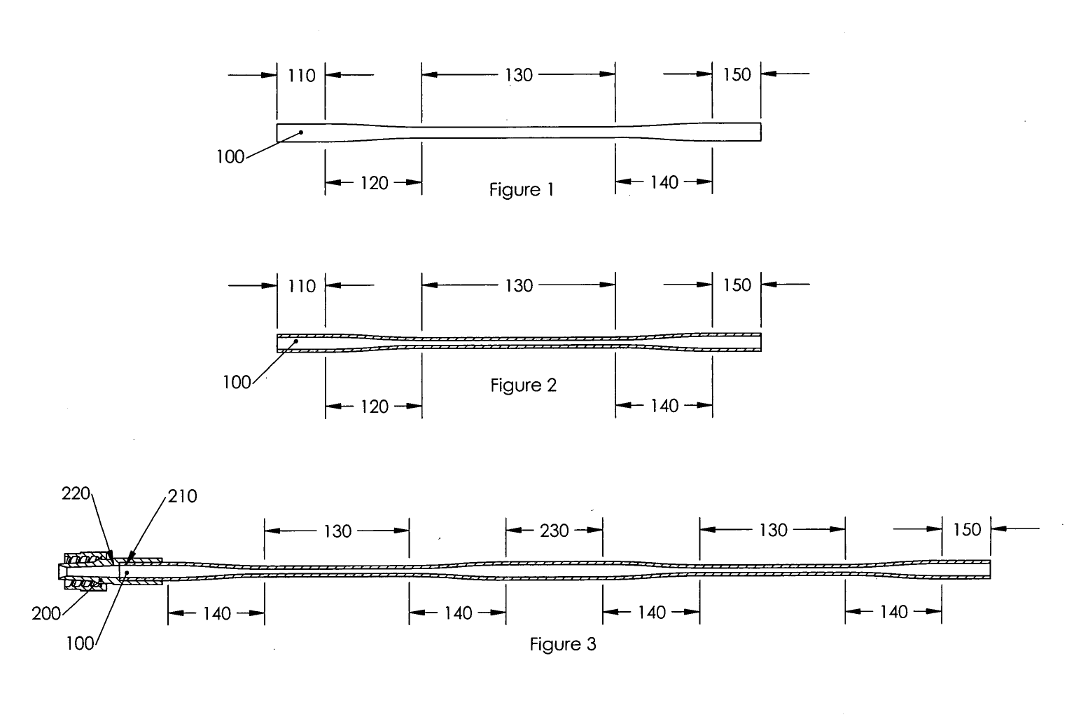

FIG. 1 illustrates a tapered tubing 100 that may be applied as a blood passage for a extracorporeal blood circuit, such as an ultrafiltration system. The blood tubing may be formed of a biocompatible plastic material such as a medical grade PVC material. The tubing is flexible transparent, and may be use in the circuit as a blood passage extending between (for example): a withdrawal catheter and a filter or sensor on the circuit, the filter (or sensor) and an infusion catheter, and between other components of the circuit.

The tapered tube 100 includes large diameter tube sections 110, 150 at the tube ends, a thin constant diameter middle tube end section 130, and tapered tube sections 120, 140 between the large and thin sections. The end sections 110 and 150 have a relatively large diameter that are sized to match the circuit components to which they are to be connected, such as a leur connector 200 (FIG. 3). The tube end sections 110 and 150 are shown as having the same length and diameter. However, the length and diameter of the end sections is influence by the particular design and the components to which the end sections are to be connected. Moreover, the end sections may be uniform in size and shape, but alternatively may have different lengths, diameters and shape to connect with mating circuit components. Additionally, it is not required that the tube diameter increase at both ends. The tapered tube 100 may have only one end section with an increased diameter. One end of the tapered tube could increase to a large diameter end section 110 and the other end may have the same or smaller diameter as does the center section 130.

The length of the narrow constant diameter section 130 of the tapered tube may be at least 0.5 inch to facilitate tubing manufacturing. The maximum length of the narrow constant diameter is a matter of design choice and may involve considerations such as flow resistance of the tube. Further, the tube may have multiple narrow tube sections 130 between large diameter ends 110, 150 and a large diameter middle section 230 (FIG. 3).

The transition tube sections 120 and 140 are tapered such that the tubing diameter is reduced during these sections. The outer and inner diameters of the tube may be reduced proportionally in the transition sections. The lengths of transition sections 120, 140 are determined based on manufacturing considerations and the desired diametric change of the tubing. The tubing diametric change is preferably smooth and continuous, and without ledges or steps in the inside blood passage surface. The transition sections preferably do not each exceed one foot.

The constant diameter narrow tube section(s) 130 generally has the thinnest diameter of the tube 100. Preferably, the inner diameter of the middle tube section 130 is at least 0.060 inch when blood flow is 40 milliliter per minute (ml/m) so as to avoid excessive flow resistance in the tube which could affect the ability of the blood circuit controller to detect disconnects. The length of the center section may be maximized to minimize the circuit volume. In contrast the lengths of the end sections 110, 150 and transition sections 120, 140 may be minimized while allowing sufficient lengths to connect to the mating components and provide a transition between the large and small diameter sections.

FIG. 2 illustrates a cross section of the tapered tubing 100. The inner and outer diameters of the tubing change in relative equal amounts along the length of the tube. By maintaining the proportion of the inner and outer diameters in the tube, the tube wall thickness remains relatively constant along the length of the tube. In addition, FIG. 2 shows that the inner blood passage surface of the tube 100 is smooth and substantially devoid of steps and discontinuities. By way of example, the end section of the tube has an outer diameter of 0.19 inch and an inner diameter of 0.13 inch, and the center section has an outer diameter of 0.16 inch and an inner diameter of 0.10 inch. Thus, by way of example, the diameter of the tubing may increase in a range of 20 percent to 30 percent from the thin diameter center section to the large diameter end tube section.

FIG. 3 illustrates the tapered tubing 100 connected to a male leur connector 200, which is a conventional connection device used in a blood circuit. At the end section, the inner diameter of the tubing 210 closely matches the inner diameter of the blood passage of the leur connector 220, such that there is no or only a minimal step between the leur connector and the tubing. A male leur connector 200 with a solvent bond port is shown, but other components and joining techniques could also apply. The port mates to the end of the tapered tubing and a solvent bonds the lure connector to the tubing.

Reducing the tubing diameter in the narrow section 130 of the tube reduces the internal volume of the tube 100 to a smaller volume than if the tube diameters were constant throughout the entire length of the tube. A smaller volume of the tapered tube results in a lower blood residence time within the tube (and hence the circuit including the tube), for a given blood flow rate. A lower blood residence time reduces the potential for blood clot formation before the extracorporeal blood is infused into the patient. Additionally, a lower tubing volume presents less risk of blood loss to the patient. If the circuit were to clot and become inoperative before the blood contained within could be returned to the patient, the patient would suffer lower blood loss with a lower volume circuit.

A constant small diameter tube and blood passage throughout the entire circuit would minimize the volume of the circuit. However, a larger tubing diameter is often needed to connect the tubing to blood circuit components, such as to a leur connector or sensor transducer housing. These components may have blood ports with relatively large diameters and would not easily interface directly with a small diameter tube. By expanding tubing at the end(s) to connect to a component, a direct connection can be made between a small-diameter tube and component without need for a diameter transition adopter. Additionally, tapering the tubing at the end(s) can simplify the circuit manufacturing process by allowing for common bonding processes and assembly equipment for all of the circuit components, regardless of the diameters of the blood ports to the components.

In some situations, it may be desirable to increase the diameter of the tube in the middle section of the tubing. While this does not reduce the circuit volume, it can allow for direct connection to other components without the need of adaptors. For example, it may be advantageous to insert a section 230 of the tubing between the track and rollers of a peristaltic pump. The large diameter section of tubing inserted into the pump may even be of a diameter larger than is required for the connecting components on the ends of the tube. Utilizing larger diameter tubing in the pump section 230 allows the pump to operate at a lower speed, reduce heat build within the pump, and reduce the risk of blood damage. In another example, the tapered tubing may have a end with a small diameter for coupling to a small diameter connecting component. Tapered tubing with a large diameter end and an opposite small diameter end does not unnecessarily restrict blood flow and reduces the extracorporeal blood volume of the circuit.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. An extracorporeal blood tube comprising:
a first end of the tube having a first inside diameter,
a narrow section of the tube having a second inside diameter substantially narrower than the first inside diameter, and
a tapered tube transition section between the first end and the narrow section.

2. An extracorporeal blood circuit as in claim 1 wherein the narrow section is a center tube section.

3. An extracorporeal blood circuit as in claim 1 further comprising a second end having an inside diameter substantially greater than the second inside diameter.

4. An extracorporeal blood circuit as in claim 1 wherein the transition section is no greater than twelve inches in length.

5. An extracorporeal blood circuit as in claim 1 wherein a wall thickness of the tube is substantially constant along an entire length of the tube.

6. An extracorporeal blood circuit as in claim 1 wherein the second inside diameter is at least 0.060 inch.

7. An extracorporeal blood circuit as in claim 1 further comprising a pump section having a third inside diameter larger than the first inside diameter.

8. An extracorporeal blood circuit as in claim 1 further comprising a pump section having a third inside diameter larger than the second inside diameter.

9. An extracorporeal blood circuit as in claim 1 wherein the first end is connectable to a connector.
